(19) United States
(12) Patent Application Publication
(10) Pub. No.: US 2021/0177008 A1
(43) Pub. Date: Jun. 17, 2021

(54) BODY WEIGHT CONTROL METHOD FOR PET

(71) Applicant: Unicharm Corporation, Ehime (JP)

(72) Inventors: Junya Sakoda, Itami-shi (JP); Fumisato Yoshiga, Itami-shi (JP); Tomoko Sakaguchi, Itami-shi (JP)

(73) Assignee: Unicharm Corporation, Ehime (JP)

(21) Appl. No.: 17/117,684

(22) Filed: Dec. 10, 2020

(30) Foreign Application Priority Data
Dec. 17, 2019 (JP) 2019-227592

(51) Int. Cl. A23K 20/147; A23K 50/45; A23K 50/42; A23K 10/30

(52) U.S. Cl. CPC A23K 20/147; A23K 10/30; A23K 50/42; A23K 50/45

(57) ABSTRACT

Provided is a body weight control method for a pet, including a body weight reduction step of feeding pet food, which has a moisture content of less than 25%, and a protein content of 20% by mass or more in terms of dry matter and a soybean-derived ingredient content of 5% to 30% by mass in terms of dry matter with respect to the total amount of pet food, to a pet daily for a period of 20 days to 100 days in a feeding amount that meets a daily energy requirement of 70×kg BW^{0.75}, in which a body fat percentage of the pet immediately after completion of the body weight reduction step is reduced by 5% or more with respect to a body fat percentage of the pet at a start of the body weight reduction step.

### BACKGROUND OF THE INVENTION

### Field of the Invention

The present invention relates to a body weight control method for a pet.

Priority is claimed on Japanese Patent Application No. 2019-227592, filed on Dec. 17, 2019, the content of which is incorporated herein by reference.

### Description of Related Art

In recent years, obesity of pets has become a problem. Obesity is a physical condition in which energy intake is higher than energy consumption and thus body fat is excessively accumulated. Excessive accumulation of body fat may cause various diseases such as diabetes, hypertension, hyperlipidemia, and arteriosclerosis.

Therefore, in order to maintain the health of pets, it is important to appropriately control the body fat mass to prevent or eliminate obesity. Further, in a case of reducing the body fat mass of pets, it is important to prevent reduction of the muscle mass of pets.

In a case where the feeding amount (intake) of pet food is reduced to an amount that does not meet the daily energy requirement for a certain period of time for the purpose of reducing the body weight, the body weight of a pet will be reduced during that period of time. However, in a case where the feeding amount (intake) of pet food is reduced, since the feeding amount (intake) of proteins is also reduced along with the reduction of the feeding amount of pet food, there is a concern that not only the body fat mass but also the muscle mass are reduced. In a case where the muscle mass is reduced, the basal metabolism is reduced. Accordingly, in a case where the feeding amount (intake) of pet food is returned to the amount that meets the daily energy requirement after the body weight of a pet is reduced, the body weight of the pet is highly likely to be returned to or increased than the body weight before the reduction of the body weight.

Published Japanese Translation No. 2015-530880 of the PCT International Publication describes a method of controlling and reducing the body weight of a cat raised as a pet.

### SUMMARY OF THE INVENTION

However, in the method of controlling and reducing the body weight disclosed in Published Japanese Translation No. 2015-530880 of the PCT International Publication, two kinds of pet foods which are a calorie-reduced diet and a calorie-maintaining diet with different calories are used. Therefore, it is complicated for a pet breeder to handle the pet food.

Further, in the method of controlling and reducing the body weight disclosed in Published Japanese Translation No. 2015-530880 of the PCT International Publication, since the calorie reduction period is set to a short period of 3 weeks at the longest, there is a high possibility (possibility of weight rebound) that the body weight after the period is returned to or increased than the body weight at the start of the calorie reduction period even though the body weight is reduced during the calorie reduction period.

The present invention has been made in consideration of the above-described circumstances, and an object thereof is to provide a body weight control method using pet food for controlling the body weight that is capable of maintaining the muscle mass while reducing the body fat mass by means of the pet food containing vegetable-derived protein source ingredients.

The present invention includes the following aspects.

(1) A body weight control method for a pet, including: a body weight reduction step of feeding pet food, which has a moisture content of less than 25%, and a protein content of 20% by mass or more in terms of dry matter and a soybean-derived ingredient content of 5% to 30% by mass in terms of dry matter with respect to a total amount of the pet food, to a pet daily for a period of 20 days to 100 days in a feeding amount that meets a daily energy requirement of 70×kg BW^{0.75}, in which a body fat percentage of the pet immediately after completion of the body weight reduction step is reduced by 5% or more with respect to a body fat percentage of the pet at a start of the body weight reduction step.

(2) The body weight control method for a pet according to (1), further including: a body weight maintenance step of feeding the pet food to the pet daily for a period of 20 days to 100 days in a feeding amount that meets a daily energy requirement of 95×kg BW^{0.75} after the body weight reduction step, in which a body fat percentage of the pet immediately after completion of the body weight maintenance step is within ±25% of the body fat percentage of the pet immediately after the completion of the body weight reduction step.

(3) The body weight control method for a pet according to (1) or (2), in which a lean body mass rate of the pet immediately after the completion of the body weight reduction step is increased by 9% or more with respect to a lean body mass rate of the pet at the start of the body weight reduction step.

(4) The body weight control method for a pet according to (3), further including: a body weight maintenance step of feeding the pet food to the pet daily for a period of 20 days to 100 days in a feeding amount that meets a daily energy requirement of 95×kg BW^{0.75} after the body weight reduction step, in which a lean body mass rate of the pet immediately after the completion of the body weight maintenance step is increased by 12% or more with respect to the lean body mass rate of the pet at the start of the body weight reduction step.

(5) The body weight control method for a pet according to any one of (1) to (4), in which a (lean body mass)/(body fat mass) of the pet immediately after the completion of the body weight reduction step is increased by 30% or more with respect to a (lean body mass)/(body fat mass) of the pet at the start of the body weight reduction step.

(6) The body weight control method for a pet according to (5), further including: a body weight maintenance step of feeding the pet food to the pet daily for a period of 20 days to 100 days in a feeding amount that meets a daily energy requirement of 95×kg BW^{0.75} after the body weight reduction step, in which the (lean body mass)/(body fat mass) of the pet immediately after the completion of the body weight maintenance step is increased by 50% or more with respect to the (lean body mass)/(body fat mass) of the pet at the start of the body weight reduction step.

(7) A body weight control method for a pet, including: a body weight reduction step of feeding pet food, which has a moisture content of less than 25%, and a protein content of 20% by mass or more in terms of dry matter and a soybean-derived ingredient content of 5% to 30% by mass in terms of dry matter with respect to a total amount of pet food, to a pet daily for a period of 20 days to 100 days in a feeding amount that meets a daily energy requirement of 70×kg BW^{0.75}, in which a reduction rate of a body weight of the pet for 7 consecutive days in the body weight reduction step is 1.5% or less.

According to the present invention, it is possible to provide the body weight control method using pet food for controlling the body weight that is capable of reducing the loss of muscle mass while reducing the body fat mass by means of the pet food containing vegetable-derived protein source ingredients.

### DETAILED DESCRIPTION OF THE INVENTION

In the present specification, the term “pets” indicate animals that have been bred by people. In a narrower sense, pets are animals being cared for by their owners. Further, “pet food” indicates feed for pets. The pet food according to the present invention can be sold as “animal feed” or “animal food”. The pet food of the present invention is eaten by various animals, but is preferred by cats and dogs, particularly dogs. Pet food can be classified into comprehensive nutritional diets provided as regular diets, between-meal snacks provided as snacks, therapeutic diets having particular ingredients used for pets with diseases, and other purpose diets, and it is appropriate that the pet food used in a body weight control method of the present invention is a comprehensive nutritional diet.

In the present specification, the “body condition score (BCS)” indicates an index showing the degree of obesity, and the standard of European Pet Food Industry Federation (FEDIAF) is used. In a case of BCS, the degree of obesity is evaluated on a 9-point scale by a veterinarian by confirming the pet's chin, around the neck, the ribs, the belly, the waist, the manner of how fat is formed on the tail, and whether constriction occurs, visually and by palpation.

In FEDIAF, according to BCS, a case of “emaciated” is evaluated as 1, a case of “very thin” is evaluated as 2, a case of “thin” is evaluated as 3, a case of “slightly underweight” is evaluated as 4, a case of “ideal (ideal weight (normal))” is evaluated as 5, a case of “slightly overweight” is evaluated as 6, a case of “overweight” is evaluated as 7, a case of “obese” is evaluated as 8, and a case of “grossly obese” is evaluated as 9.

### First Embodiment

<Body Weight Control Method for Pet>

A body weight control method for a pet according to a first embodiment is a body weight control method including a body weight reduction step of feeding pet food, which has a moisture content of less than 25%, and a protein content of 20% by mass or more in terms of dry matter and a soybean-derived ingredient content of 5% to 30% by mass in terms of dry matter with respect to the total amount of pet food, to a pet daily for a period of 20 days to 100 days in a feeding amount that meets a daily energy requirement of 70×kg BW^{0.75}, in which the body fat percentage of the pet immediately after completion of the body weight reduction step is reduced by 5% or more with respect to the body fat percentage of the pet at a start of the body weight reduction step.

<<Pet Food>>

In the body weight control method for a pet according to the present embodiment, pet food which has a moisture content of less than 25%, and a protein content of 20% by mass or more in terms of dry matter and a soybean-derived ingredient content of 5% to 30% by mass in terms of dry matter with respect to the total amount of pet food is used.

The pet food used in the present embodiment is pet food for controlling the body weight, which is used for controlling the body weight while reducing the loss of muscle mass.

The pet food used in the present embodiment is pet food that can reduce the body fat mass and also reduce the loss of muscle mass. That is, the pet food of the present embodiment is pet food that can maintain the lean body mass rate while reducing the body fat percentage. In this manner, a breeder can control the body weight of a pet by feeding the pet food daily to the pet.

<<Moisture Content>>

The pet food used in the present embodiment has a moisture content of less than 25%.

Pet food is largely classified into dry food having a moisture content of approximately 10%, soft food having a moisture content of approximately 15% to 35%, and wet food having a moisture content of approximately 80% according to the classification based on the moisture content.

In a case where the moisture content of the pet food used in the present embodiment is less than 25%, the storage period of the pet food is relatively long, and the odor of the pet food can be suppressed. Therefore, the breeder can easily handle the pet food.

The moisture content of the pet food used in the present embodiment is preferably less than 20%, more preferably less than 15%, and still more preferably less than 10%.

<<Protein Content>>

The pet food used in the present embodiment has a protein content of 20% by mass or more in terms of dry matter with respect to the total amount of the pet food. With the above-described configuration, it is possible to feed a pet with a larger amount of proteins necessary for muscle synthesis while satisfying the nutrients of pet food as a comprehensive nutritional diet. The pet food used in the present embodiment has a protein content of preferably 24% by mass or more in terms of dry matter and more preferably 31% by mass or more in terms of dry matter with respect to the total amount of the pet food.

Protein source ingredients are largely classified into animal protein source ingredients derived from animals and vegetable protein source ingredients derived from vegetables, and the proteins in the pet food are derived from these ingredients. Specific examples of the animal protein source ingredients include chicken meal, pork meal, and beef meal. The animal protein source ingredients are difficult to be adjusted as the protein source ingredients because inedible parts (bones containing a large amount of minerals, internal organs, and the like) are subjected to a heat treatment, degreased, dried, and powdered.

Specific examples of the vegetable protein source ingredients include soybean-derived ingredients, corn-derived ingredients such as corn gluten meal, and wheat-derived ingredients such as wheat gluten.

<<Soybean-Derived Ingredients>>

The pet food used in the present embodiment has a soybean-derived ingredient content of 5% to 30% by mass in terms of dry matter with respect to the total amount of the pet food. Specific examples of the soybean-derived ingredients include extruder-treated soybeans, puffed soybeans, puffed dehulled soybeans, soybean flour, fermented soybean meal, isolated soybean protein, and defatted soybeans. Among the soybean-derived ingredients, it is more preferable to use the fermented soybean meal and the isolated soybean protein and particularly preferable to use the isolated soybean protein for the pet food used in the present embodiment.

<<Isolated Soybean Protein>>

The isolated soybean protein is obtained by degreasing soybeans, adding an acid to soy milk obtained by extraction with water to form whey and curd, centrifuging the curd portion or separating the curd portion with a filter, and neutralizing, drying, and grinding the resultant. The isolated soybean protein has a higher protein content than that of concentrated soybean protein. 99.9% of the isolated soybean protein is formed of protein components, and the isolated soybean protein contains almost no protease inhibitor which is a protein digestion inhibitor. The isolated soybean protein is displayed as “soybean protein” in the ingredient indication of the product, and the name of the ingredient indication is different from that of other soybean products (including defatted soybeans). The amino acid balance of the “soybean protein” is similar to that of animal-derived protein, but contains less minerals than those of the animal protein source ingredients and almost no oil and fat content, and thus the nutrition is easily designed.

Since the isolated soybean protein contains almost no protease inhibitor, the digestion of the proteins contained in the pet food is not inhibited.

In the present embodiment, the lower limit of the amount of the isolated soybean protein is preferably 5% by mass or more in terms of dry matter. The lower limit of the amount of the isolated soybean protein is more preferably 10% by mass or more in terms of dry matter and still more preferably 12% by mass or more in terms of dry matter. The upper limit of the amount of the isolated soybean protein is preferably 30% by mass or less in terms of dry matter. The upper limit of the amount of the isolated soybean protein is more preferably 25% by mass or less in terms of dry matter and still more preferably 20% by mass or less in terms of dry matter. With the above-described configuration, the amino acid balance can be adjusted. Further, in the present embodiment, the protein content with respect to the total amount of the pet food also includes the amount of the isolated soybean protein.

<<Lipid Content>>

The pet food used in the present embodiment has a lipid content of preferably 5.5% to 30.0% by mass in terms of dry matter with respect to the total amount of the pet food.

With the above-described configuration, the body weight of the pet can be reduced without reducing the lipid content of the pet food as in the therapeutic diet. Further, with the above-described configuration, the body weight and body fat of the pet can be reduced while suppressing reduction of the lean body mass.

The pet food used in the present embodiment has a lipid content of more preferably 5.5% to 20.0% by mass and still more preferably 5.5% to 15.0% by mass in terms of dry matter with respect to the total amount of the pet food.

<<Trypsin Inhibitor>>

The pet food used in the present embodiment contains preferably 1.3 TIU/mg or less of a trypsin inhibitor in terms of dry matter with respect to the total amount of the pet food.

The trypsin inhibitor is one of the factors that inhibit an enzyme (proteolytic enzyme) that decomposes proteins and is also a factor that influences the digestibility of amino acids constituting the proteins. Specifically, proteins ingested from the oral cavity are decomposed into amino acids and peptides that can be absorbed into the body by the proteolytic enzyme and are absorbed into the body. The amino acids and peptides absorbed into the body are synthesized in muscle and are the sources of hemoglobin and hormones in the blood. Therefore, in a case where the amount of the trypsin inhibitor, which is a factor that inhibits the proteolytic enzyme, is small, since the decomposition of proteins into amino acids or peptides is not inhibited, the digestibility of proteins is increased. Due to the high digestibility of proteins, since a larger amount of amino acids required for muscle synthesis can be absorbed into the body, the body weight is easily controlled while the loss of muscle mass is reduced.

As described above, the isolated soybean protein contains almost no trypsin inhibitor. Therefore, the trypsin inhibitor contained in the pet food used in the present embodiment is derived from vegetable ingredients other than the isolated soybean protein.

The amount of the trypsin inhibitor is more preferably 1.2 TIU/mg or less in terms of dry matter and still more preferably 1.0 TIU/mg or less with respect to the total amount of the pet food.

<Body Weight Reduction Step>

The body weight control method for a pet according to the present embodiment includes a body weight reduction step of feeding the pet food to a pet daily for a period of 20 days to 100 days in a feeding amount that meets a daily energy requirement of 70×kg BW^{0.75}.

The “body weight reduction step” is a step of feeding the pet food in a feeding amount that meets a daily energy requirement of 70×kg BW^{0.75} to a pet daily for a period of 20 days to 100 days by a breeder of the pet.

By providing the body weight reduction step, the BCS of the pet can be reliably reduced to a scale of 4 to 6 while the muscle mass is maintained.

The period of the body weight reduction step (hereinafter, referred to as a “body weight reduction step period”) is more preferably in a range of 30 days to 100 days and still more preferably in a range of 50 days to 100 days.

<Body Weight Maintenance Step>

It is preferable that the body weight control method for a pet according to the present embodiment further includes a body weight maintenance step of feeding the pet food to the pet daily for a period of 20 days to 100 days in a feeding amount that meets a daily energy requirement of 95×kg BW^{0.75} after the body weight reduction step.

The “body weight maintenance step” is a step of feeding the pet food in a feeding amount that meets a daily energy requirement of 95×kg BW^{0.75} to the pet daily for a period of 20 days to 100 days by a breeder of the pet.

By providing the body weight maintenance step, the body weight and the body fat percentage of the pet whose BCS has reached a scale of 4 to 5 in the body weight reduction step are maintained in a certain range, and thus it is possible to prevent the body weight of the pet from being returned to the body weight before the body weight reduction step or being increased than the body weight before the body weight reduction step. That is, it is possible to prevent weight rebound of pets with a tendency to be obese or obese pets having a BCS of 7 to 9, after dieting.

The period of the body weight maintenance step (hereinafter, referred to as a “body weight maintenance step period”) is more preferably in a range of 30 days to 100 days and still more preferably in a range of 50 days to 100 days.

<<Feeding Amount>>

The feeding amount of pet food (g/day) is calculated for each individual based on Calculation Equation (1).

Feeding amount=DER/metabolic energy of pet food (kcal/g) Equation (1)

DER (daily energy requirement (kcal/day)) is calculated by multiplying a coefficient α set for each of the body weight reduction step period and the body weight maintenance step period by the body weight (kgBW) in terms of kg to the power of 0.75 based on Equation (2).

DER=α×kg BW^{0.75} Equation (2)

In the calculation of the feeding amount during the body weight reduction step period, the coefficient α in Equation (2) is set to “70”. In the calculation of the feeding amount during the body weight maintenance step period, the coefficient α in Equation (2) is set to “95”. Equation (2) and the coefficient α set for each of the body weight reduction step period and the body weight maintenance step period are introduced in FEDIAF Nutritional Guidelines 2017 (p. 67).

<<Body Fat Percentage>>

The “body fat percentage” indicates a value obtained by dividing the body fat mass of an individual pet by the total body weight of the individual pet. For example, the body fat percentage can be measured using a “body fat percentage meter for a health lab dog” that employs a bioimpedance method in addition to DEXA and a heavy water dilution method.

In the body weight control method for a pet according to the present embodiment, the body fat percentage of the pet immediately after the completion of the body weight reduction step is reduced by 5% or more with respect to the body fat percentage of the pet at the start of the body weight reduction step.

With the above-described configuration, the body fat percentage of the pet immediately after the completion of the body weight reduction step can be reduced by 5% or more with respect to the body fat percentage of the pet at the start of the body weight reduction step.

In a case where the body weight control method for a pet according to the present embodiment further includes the body weight maintenance step after the body weight reduction step, it is preferable that the body fat percentage of the pet immediately after the completion of the body weight maintenance step is within ±25% of the body fat percentage of the pet immediately after the completion of the body weight reduction step. With the above-described configuration, it is possible to prevent the weight rebound in which the body fat percentage rapidly increases from the value before the body weight reduction step.

<<Lean Body Mass Rate>>

The “lean body mass rate” indicates the percentage of those other than the body fat mass in the total body weight of an individual pet and also indicates a value obtained by setting the total body weight of the individual pet as 100% and performing calculation by subtracting the body fat percentage from 100%.

Lean body mass rate (%)=100%−body fat percentage (%)

Since the body fat percentage decreases as the lean body mass rate increases, it is preferable that the value of the lean body mass rate is high.

In the body weight control method for a pet according to the present embodiment, it is preferable that the lean body mass rate of the pet immediately after the completion of the body weight reduction step is increased by 9% or more with respect to the lean body mass rate of the pet at the start of the body weight reduction step.

With the above-described configuration, the lean body mass rate of the pet immediately after the completion of the body weight reduction step can be increased by 9% or more with respect to the lean body mass rate of the pet at the start of the body weight reduction step.

In a case where the body weight control method for a pet according to the present embodiment further includes the body weight maintenance step after the body weight reduction step, it is preferable that the lean body mass rate of the pet immediately after the completion of the body weight maintenance step is increased by 12% or more with respect to the lean body mass rate of the pet at the start of the body weight reduction step.

With the above-described configuration, it is possible to increase the lean body mass rate and the muscle mass while suppressing an increase in the body fat percentage, that is, suppressing the weight rebound.

<<(Lean Body Mass)/(Body Fat Mass)>>

The “(lean body mass)/(body fat mass)” indicates a value calculated by dividing the lean body mass of an individual pet by the body fat mass of the individual pet.

The “body fat mass” is the amount of fat in the body of an individual pet and can be calculated by multiplying the body weight by the body fat percentage.

The “lean body mass” is an index related to the body weight obtained by subtracting the body fat mass from the total body weight of an individual pet and is acquired by the following calculation equation.

Lean body mass (kg)=total body weight (kg)−body fat mass (kg)

In the body weight control method for a pet according to the present embodiment, it is preferable that the (lean body mass)/(body fat mass) of the pet immediately after the completion of the body weight reduction step is increased by 30% or more with respect to the (lean body mass)/(body fat mass) of the pet at the start of the body weight reduction step.

With the above-described configuration, the (lean body mass)/(body fat mass) of the pet immediately after the completion of the body weight reduction step can be increased by 30% or more with respect to the (lean body mass)/(body fat mass) of the pet at the start of the body weight reduction step.

In a case where the body weight control method for a pet according to the present embodiment further includes the body weight maintenance step after the body weight reduction step, it is preferable that the (lean body mass)/(body fat mass) of the pet immediately after the completion of the body weight maintenance step is increased by 50% or more with respect to the (lean body mass)/(body fat mass) of the pet at the start of the body weight reduction step.

With the above-described configuration, it is possible to increase the lean body mass and the muscle mass while suppressing an increase in body fat mass, that is, suppressing the weight rebound.

In the pet food used in the present embodiment, the composition of ingredients is not particularly limited as long as the pet food is a comprehensive nutritional diet that meets the nutritional dietary standards. As a pet food, it is preferable to set the composition of ingredients so as to satisfy the nutritional composition of food grains and obtain satisfactory formability. The pet food used in the present embodiment can be more preferably used as pet food for dogs.

[Ingredients]

In the pet food used in the present embodiment, the ingredients are not limited as long as the above-described configuration is employed. Known ingredients can be used in the production of pet food.

Examples of powder ingredients include cereals (such as corn, wheat, rice, barley, oats, and rye), beans (such as defatted soybeans and whole soybeans), starch (such as wheat starch, corn starch, rice starch, potato starch, tapioca starch, sweet potato starch, and sago starch), vegetable protein source ingredients (such as corn gluten meal and wheat gluten), meat (such as chicken, beef, pork, venison, meals (such as chicken meal, pork meal, beef meal, and mixed meal thereof)), seafood (such as fish and meals (fish meal)), vegetables, and powdery additives (such as vitamins, minerals, amino acids, flavoring ingredients, fibers, colorants, and palatability enhancers). The meals indicate powder products obtained by compressing and finely grinding meat or seafood.

Examples of the palatability enhancers include animal ingredient extract, vegetable ingredient extract, yeast extract (brewer's yeast extract, baker's yeast extract, or torula yeast extract), and dried yeast (brewer's yeast, baker's yeast, or torula yeast).

The composition of the ingredients is not particularly limited. It is preferable to set the composition of the ingredients such that the nutritional composition of food grains to be obtained is satisfied and satisfactory formability is obtained.

As an example of the composition, the pet food contains 40% to 75% by mass of cereals; 10% to 25% by mass of meat; 5% to 15% by mass of seafood; 2% to 5% by mass of vitamins and minerals; 2% to 20% by mass of fats and oils; and other remaining components, and the total content of the pet food is 100% by mass.

As liquid ingredients to be added to powder ingredients, liquid ingredients such as water, fats and oils, liquid sugar, a palatability enhancer solution, a flavoring agent, and a colorant can be used as necessary. Further, puffed grains are dried and may be coated with liquid ingredients (coating agent) containing fats and oils, a seasoning, a palatability enhancer, a flavoring agent, and the like.

The fats and oils may be vegetable fats and oils or animal fats and oils (chicken oil, pork fat (lard), beef tallow (fat), milk fat, and the like). The coating agent contains preferably animal fats and oils and particularly preferably beef tallow.

[Shape and Size]

The shape of the food grains constituting the pet food used in the present embodiment is not particularly limited as long as the grains have a shape suitable for the pet to eat the pet food. For example, all shapes such as a spherical shape, a polygonal shape, a columnar shape, a donut shape, a plate shape, an ellipsoidal shape (go stone shape), and a clover shape can be applied. Further, in regard to the size of food grains, the food grains may be small such that a pet can fit all of it in the mouth or may be large such that a pet can bite into the food plural times.

For example, in regard to the size of food grains, both the shortest diameter and the longest diameter are preferably in a range of 3 to 30 mm, more preferably in a range of 6 to 16.5 mm, and still more preferably in a range of 7 to 13 mm.

### Second Embodiment

A body weight control method for a pet according to a second embodiment is a body weight control method for a pet, including a body weight reduction step of feeding pet food, which has a moisture content of less than 25%, and a protein content of 20% by mass or more in terms of dry matter and a soybean-derived ingredient content of 5% to 30% by mass in terms of dry matter with respect to the total amount of pet food, to a pet daily for a period of 20 days to 100 days in a feeding amount that meets a daily energy requirement of 70×kg BW^{0.75}, in which the reduction rate of the body weight of the pet for 7 consecutive days in the body weight reduction step is 1.5% or less.

The present embodiment is different from the above-described embodiment in terms that the reduction rate of the body weight of the pet for 7 consecutive days in the body weight reduction step is 1.5% or less.

Specifically, in the body weight control method for a pet according to the present embodiment, the reduction rate in a case where the body weight of an individual pet on the first day is compared with the body weight of the individual pet on the seventh day in the 7 consecutive days of the body weight reduction step period is 1.5% or less. With the above-described configuration, the body weight of the pet can be reduced at a constant rate and the loss of muscle mass can be reduced. Further, in a case where the reduction rate is greater than 1.5%, the body weight is rapidly reduced, and the loss of muscle mass is significant.

Further, it is preferable that the body weight of the pet is gradually reduced during the body weight reduction step period.

The lower limit of the reduction rate of the body weight of the pet for 7 consecutive days in the body weight reduction step is preferably 1.0% or more. In a case where the reduction rate is less than 1.0%, the body weight reduction step period is extended, and thus it takes time and effort to reach a preferable body weight.

The reduction rate of the body weight of the pet for 7 consecutive days in the body weight reduction step is particularly preferably 1.3%.

### Modified Example

Hereinbefore, the preferred embodiments of the present invention have been described, but the present invention is not limited to these embodiments. Additions, omissions, substitutions, and other modifications of the configurations can be made within a range not departing from the gist of the present invention. The present invention is not limited by the description above, but by the range of the appended claims.

For example, the configuration of the second embodiment can be applied to the configuration of the first embodiment.

<Method of Producing Pet Food>

As a method of producing the pet food used in the present embodiment, a known method can be used, and the production method is not particularly limited as long as the configuration of the present embodiment is employed. Examples of the known method include a method of producing pet food, including a granulating step, a drying step, and a coating step in order.

[Granulating Step]

The granulating step is a step of granulating an ingredient mixture to obtain grains.

The granulating step may be performed according to a method of mixing ingredients to obtain an ingredient mixture and forming (granulating) the ingredient mixture into granules.

Specifically, the granulating step may be performed according to a method of producing grains (puffed grains) using an extruder.

As the method of producing grains using an extruder, for example, a method described in “Clinical Nutrition of Small Animals, 5th edition” (edited by Michael S. Hand, Craig D. Thatcher, Rebecca L. Remillard, Philip Roudebusg, and Bruce J. Novotny, published by Mark Morris Associates; 2014; p. 209 to 215) can be applied.

[Drying Step]

The drying step is a step of drying the grains obtained by the granulating step described above.

Examples of the method of drying the grains include known methods such as a method of naturally drying the grains, a method of blowing warm air to dry the grains, a method of drying the grains under reduced pressure, and a method of freeze-drying the grains. Among these drying methods, from the viewpoints of inactivating the protease inhibitor and increasing the protein digestibility, the method of blowing warm air to dry the grains is preferable.

[Coating Step]

The coating step is a step of coating the food grains obtained by the drying step described above with a coating agent containing crude beef tallow, a seasoning, or a flavoring agent.

The method of coating the food grains is not particularly limited, and for example, a vacuum coating method can be used. The vacuum coating method is a method of reducing the pressure in a state in which heated grains and fats and oils are brought into contact with each other or attached to each other. The coating agent may be in the form of a liquid or powder. By coating the grains, the palatability (biting property) of a pet can be improved.

### Examples

<Production of Pet Food>

Ingredients constituting a comprehensive nutritional diet pet food were mixed with the composition listed in Table 1. The obtained ingredient mixture was put into an extruder and subjected to a heat treatment in a temperature range of 100° C. to 140° C. for 1 to 5 minutes while being kneaded so that starch components were pregelatinized, and the mixture was allowed to be puffed simultaneously with extrusion and granulation of the mixture in a granular shape at an outlet of the extruder, thereby obtaining puffed grains. The obtained puffed grains were subjected to a drying treatment at 125° C. for 15 minutes using a dryer, thereby obtaining pet food 1 used in the test examples.

TABLE 1

| | Pet food 1 |
|---|---|
| Animal protein | 17.5% |
| Isolated soybean protein | 10.0% |
| Cereals | 57.1% |
| Cellulose | 3.0% |
| Beet pulp | 2.0% |
| Animal fats and oils | 7.0% |
| Vitamins and minerals | 2.4% |
| Other components | 1.0% |
| Total | 100.0% |

The “other components” in Table 1 were components derived from palatability enhancers such as animal ingredient extract, vegetable ingredient extract, and yeast extract.

Table 2 shows the general component analysis results of the pet food 1 used in the test examples.

TABLE 2

| | Pet food 1 |
|---|---|
| Moisture | 8.2% |
| Protein | 31.2% |
| Lipid | 13.0% |
| Crude fiber | 2.6% |
| Ash | 4.5% |
| Calories | 361.5 kcal/100 g |
| Trypsin inhibitor | 0.3 TIU/mg |

[Body Weight Control]

<<Body Weight Reduction Step>>

The body weight reduction step was set to be performed in a period of the test start day (day 0) to the 84th day (the start day of the first week to the final day of the 12th week).

The feeding amount of the pet food 1 in the body weight reduction step was calculated based on Equation (1) by setting the coefficient α in Equation (2) to “70”.

<<Body Weight Adjustment Step>>

The body weight adjustment step was set to be performed in a period of the 85th day to the 119th day (the start day of the 13th week to the final day of the 17th week) from the test start day.

The feeding amount of the pet food 1 in the body weight adjustment step was calculated based on Equation (1) by setting the coefficient α in Equation (2) for each individual pet.

The reason why the “body weight adjustment step” in which the coefficient α was set for each individual pet was provided between the body weight reduction step and the body weight maintenance step is to clarify a change in evaluation in the body weight maintenance step after the adjustment period by adjusting BCS of all individual pets to be in a range of 4 to 5 so that a variation in BCS that was generated on the final day (84th day) of the body weight reduction step was eliminated. Further, in an actual consumption site, the body weight adjustment step may not be provided in a case where BCS has been adjusted to be in a range of 4 to 5.

<<Body Weight Maintenance Step>>

The body weight maintenance step was set to be performed in a period of the 120th day to the 209th day (the start day of the 17th week to the final day of the 29th week) from the test start day.

The feeding amount of the pet food 1 in the body weight maintenance step was calculated based on Equation (1) by setting the coefficient α in Equation (2) to “95”.

<Body Weight Control Evaluation>

The body weight, the body fat percentage, the lean body mass rate, and LBM/Fat were used for the evaluations of the body weight control. The measurement dates of the body weight and the body fat percentage were set to the test start day (day 0), the 28th day, the 56th day, the 84th day, the 119th day, the 147th day, the 175th day, and the 203rd day from the test start day. The lean body mass rate and LBM/Fat were calculated based on the measured values of the body weight and the body fat percentage on the above-described measurement dates.

<<Body Weight>>

The total body weight of the individual pet including the hair was measured using GP100K (manufactured by A & D Co., Ltd.) and defined as the body weight (kg).

<<Body Fat Percentage>>

The body fat percentage was measured using a “body fat percentage meter for a health lab dog” that employs a bioimpedance method.

<<Lean Body Mass Rate>>

The lean body mass rate was calculated by setting the body weight of the individual pet as 100% and performing calculation by subtracting the body fat percentage from 100%.

Lean body mass rate (%)=100%−body fat percentage (%)

Since the body fat percentage decreases as the lean body mass rate increases, it is preferable that the value of the lean body mass rate is high.

<<(Lean Body Mass)/(Body Fat Mass) (LBM/Fat)>>

LBM/Fat was defined as a value obtained by dividing the lean body mass (LBM) by the body fat mass (Fat). The lean body mass (LBM) was calculated by multiplying the body weight by the lean body mass rate, and the body fat mass (Fat) was calculated by multiplying the body weight by the body fat percentage.

The evaluation of the body weight is listed in Table 3, the evaluation of the body fat percentage is listed in Table 4, the evaluation of the lean body mass rate is listed in Table 5, and the evaluation of LBM/Fat is listed in Table 6.

TABLE 3

| Test | Reduction | | | | Adjustment | Maintenance | | |
|---|---|---|---|---|---|---|---|---|
| Example | period | | | | period | period | | |
| Individual | 0th | 28th | 56th | 84th | 119th | 147th | 175th | 203rd |
| No. | day | day | day | day | day | day | day | day |
| 1 | 13.8 | 12.3 | 11.6 | 10.6 | 10.0 | 9.7 | 9.6 | 9.7 |
| 2 | 13.9 | 12.6 | 11.9 | 11.4 | 11.3 | 11.4 | 11.2 | 10.9 |
| 3 | 15.0 | 13.1 | 12.3 | 11.9 | 11.8 | 11.6 | 11.6 | 11.8 |
| 4 | 15.8 | 15.2 | 14.6 | 14.0 | 13.0 | 13.7 | 13.6 | 13.7 |
| 5 | 15.3 | 13.5 | 12.9 | 12.5 | 12.4 | 12.5 | 12.2 | 12.1 |
| 6 | 15.7 | 13.7 | 12.0 | 10.8 | 11.9 | 11.3 | 10.5 | 10.4 |
| 7 | 13.3 | 11.4 | 10.7 | 10.2 | 9.8 | 9.4 | 9.2 | 9.1 |
| Average | 14.7 | 13.1 | 12.3 | 11.6 | 11.5 | 11.4 | 11.1 | 11.1 |
| Standard | 0.9 | 1.1 | 1.1 | 1.2 | 1.1 | 1.4 | 1.4 | 1.5 |
| deviation | | | | | | | | |

TABLE 4

| Test | Reduction | | | | Adjustment | Maintenance | | |
|---|---|---|---|---|---|---|---|---|
| Example | period | | | | period | period | | |
| Individual | 0th | 28th | 56th | 84th | 119th | 147th | 175th | 203rd |
| No. | day | day | day | day | day | day | day | day |
| 1 | 26.3 | 25.7 | 20.3 | 19.3 | 19.0 | 18.3 | 17.3 | 14.7 |
| 2 | 31.3 | 30.0 | 25.7 | 21.0 | 20.0 | 20.7 | 19.3 | 19.7 |
| 3 | 33.0 | 32.7 | 23.3 | 19.3 | 19.0 | 18.7 | 18.0 | 17.3 |
| 4 | 33.3 | 33.0 | 31.7 | 27.3 | 24.0 | 25.7 | 26.0 | 25.0 |
| 5 | 33.3 | 29.7 | 23.3 | 18.7 | 19.3 | 19.7 | 19.3 | 19.3 |
| 6 | 27.0 | 24.7 | 17.0 | 14.0 | 15.3 | 15.0 | 14.3 | 14.3 |
| 7 | 25.7 | 25.0 | 22.3 | 19.0 | 17.7 | 16.0 | 15.3 | 15.3 |
| Average | 30.0 | 28.7 | 23.4 | 19.8 | 19.2 | 19.1 | 18.5 | 18.0 |
| Standard | 3.3 | 3.3 | 4.2 | 3.7 | 2.4 | 3.2 | 3.5 | 3.5 |
| deviation | | | | | | | | |

TABLE 5

| Test | Reduction | | | | Adjustment | Maintenance | | |
|---|---|---|---|---|---|---|---|---|
| Example | period | | | | period | period | | |
| Individual | 0th | 28th | 56th | 84th | 119th | 147th | 175th | 203rd |
| No. | day | day | day | day | day | day | day | day |
| 1 | 73.7 | 74.3 | 79.7 | 80.7 | 81.0 | 81.7 | 82.7 | 85.3 |
| 2 | 68.7 | 70.0 | 74.3 | 79.0 | 80.0 | 79.3 | 80.7 | 80.3 |
| 3 | 67.0 | 67.3 | 76.7 | 80.7 | 81.0 | 81.3 | 82.0 | 82.7 |
| 4 | 66.7 | 67.0 | 68.3 | 72.7 | 76.0 | 74.3 | 74.0 | 75.0 |
| 5 | 66.7 | 70.3 | 76.7 | 81.3 | 80.7 | 80.3 | 80.7 | 80.7 |
| 6 | 73.0 | 75.3 | 83.0 | 86.0 | 84.7 | 85.0 | 85.7 | 85.7 |
| 7 | 74.3 | 75.0 | 77.7 | 81.0 | 82.3 | 84.0 | 84.7 | 84.7 |
| Average | 70.0 | 71.3 | 76.6 | 80.2 | 80.8 | 80.9 | 81.5 | 82.0 |
| Standard | 3.3 | 3.3 | 4.2 | 3.7 | 2.4 | 3.2 | 3.5 | 3.5 |
| deviation | | | | | | | | |

TABLE 6

| Test | Reduction | | | | Adjustment | Maintenance | | |
|---|---|---|---|---|---|---|---|---|
| Example | period | | | | period | period | | |
| Individual | 0th | 28th | 56th | 84th | 119th | 147th | 175th | 203rd |
| No. | day | day | day | day | day | day | day | day |
| 1 | 2.80 | 2.90 | 3.92 | 4.17 | 4.26 | 4.45 | 4.77 | 5.82 |
| 2 | 2.19 | 2.33 | 2.90 | 3.76 | 4.00 | 3.84 | 4.17 | 4.08 |
| 3 | 2.03 | 2.06 | 3.29 | 4.17 | 4.26 | 4.36 | 4.56 | 4.77 |
| 4 | 2.00 | 2.03 | 2.15 | 2.66 | 3.17 | 2.90 | 2.85 | 3.00 |
| 5 | 2.00 | 2.37 | 3.29 | 4.36 | 4.17 | 4.08 | 4.17 | 4.17 |
| 6 | 2.70 | 3.05 | 4.88 | 6.14 | 5.52 | 5.67 | 5.98 | 5.98 |
| 7 | 2.90 | 3.00 | 3.48 | 4.26 | 4.66 | 5.25 | 5.52 | 5.52 |
| Average | 2.37 | 2.54 | 3.41 | 4.22 | 4.29 | 4.36 | 4.57 | 4.76 |
| Standard | 0.38 | 0.41 | 0.78 | 0.95 | 0.66 | 0.85 | 0.94 | 1.01 |
| deviation | | | | | | | | |

As listed in Tables 3 to 6, it was clarified that in a case where a predetermined amount of the pet food 1 was fed to a pet in each step of the body weight reduction step and the body weight maintenance step, the body weight and the body fat mass (body fat percentage) were able to be reduced while the loss of muscle mass (lean body mass) of the pet was reduced.

According to the present invention, it is possible to provide a body weight control method using pet food for controlling the body weight that is capable of controlling the body weight while reducing the loss of muscle mass by means of the pet food containing vegetable protein source ingredients.

Hereinbefore, the preferable examples of the present invention have been described, but the present invention is not limited to these examples. Additions, omissions, substitutions, and other modifications of the configurations can be made within a range not departing from the gist of the present invention.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A body weight control method for a pet, comprising:
a body weight reduction step of feeding pet food, which has a moisture content of less than 25%, and a protein content of 20% by mass or more in terms of dry matter and a soybean-derived ingredient content of 5% to 30% by mass in terms of dry matter with respect to a total amount of the pet food, to a pet daily for a period of 20 days to 100 days in a feeding amount that meets a daily energy requirement of 70×kg BW^{0.75},
wherein a body fat percentage of the pet immediately after completion of the body weight reduction step is reduced by 5% or more with respect to a body fat percentage of the pet at a start of the body weight reduction step.

2. The body weight control method for a pet according to claim 1, further comprising:
a body weight maintenance step of feeding the pet food to the pet daily for a period of 20 days to 100 days in a feeding amount that meets a daily energy requirement of 95×kg BW^{0.75} after the body weight reduction step,
wherein a body fat percentage of the pet immediately after completion of the body weight maintenance step is within ±25% of the body fat percentage of the pet immediately after the completion of the body weight reduction step.

3. The body weight control method for a pet according to claim 1,
wherein a lean body mass rate of the pet immediately after the completion of the body weight reduction step is increased by 9% or more with respect to a lean body mass rate of the pet at the start of the body weight reduction step.

4. The body weight control method for a pet according to claim 2,
wherein a lean body mass rate of the pet immediately after the completion of the body weight reduction step is increased by 9% or more with respect to a lean body mass rate of the pet at the start of the body weight reduction step.

5. The body weight control method for a pet according to claim 3, further comprising:
a body weight maintenance step of feeding the pet food to the pet daily for a period of 20 days to 100 days in a feeding amount that meets a daily energy requirement of 95×kg BW^{0.75} after the body weight reduction step,
wherein a lean body mass rate of the pet immediately after the completion of the body weight maintenance step is increased by 12% or more with respect to the lean body mass rate of the pet at the start of the body weight reduction step.

6. The body weight control method for a pet according to claim 1,
wherein a (lean body mass)/(body fat mass) of the pet immediately after the completion of the body weight reduction step is increased by 30% or more with respect to a (lean body mass)/(body fat mass) of the pet at the start of the body weight reduction step.

7. The body weight control method for a pet according to claim 6, further comprising:
a body weight maintenance step of feeding the pet food to the pet daily for a period of 20 days to 100 days in a feeding amount that meets a daily energy requirement of 95×kg BW^{0.75} after the body weight reduction step,
wherein the (lean body mass)/(body fat mass) of the pet immediately after the completion of the body weight maintenance step is increased by 50% or more with respect to the (lean body mass)/(body fat mass) of the pet at the start of the body weight reduction step.

8. A body weight control method for a pet, comprising:
a body weight reduction step of feeding pet food, which has a moisture content of less than 25%, and a protein content of 20% by mass or more in terms of dry matter and a soybean-derived ingredient content of 5% to 30% by mass in terms of dry matter with respect to a total amount of the pet food, to a pet daily for a period of 20 days to 100 days in a feeding amount that meets a daily energy requirement of 70×kg BW^{0.75},
wherein a reduction rate of a body weight of the pet for 7 consecutive days in the body weight reduction step is 1.5% or less.
